(19) United States
(12) Patent Application Publication
(10) Pub. No.: US 2026/0159806 A1
(43) Pub. Date: Jun. 11, 2026

(54) OOCYTE MATURATION PROMOTER AND USES THEREOF

(71) Applicant: Tokunori Yamamoto, Nagoya-shi (JP)

(72) Inventors: Tokunori Yamamoto, Nagoya-shi (JP); Tetsuma Murase, Gifu-shi (JP); Takehiro Himaki, Gifu-shi (JP)

(73) Assignee: Tokunori Yamamoto, Nagoya-shi (JP)

(21) Appl. No.: 18/847,370

(22) PCT Filed: Sep. 1, 2022
(86) PCT No.: PCT/JP2022/033031

§ 371 Date: Sep. 16, 2024

(30) Foreign Application Priority Data
Mar. 18, 2022 (WO) PCT/JP2022/012703

(51) Int. Cl. C12N 5/075; C12N 5/073; C12N 5/076

(52) U.S. Cl. CPC C12N 5/0609; C12N 5/0604; C12N 5/061; C12N 2501/40

(57) ABSTRACT

The present invention relates to an oocyte maturation promoter that can be used in the fields of livestock industry and assisted reproductive technology, and use thereof. This oocyte maturation promoter contains, as an active ingredient, a filtrate of disrupted adipose tissue-derived stem cell solution.

### TECHNICAL FIELD

The present invention relates to a technology that can be used in the fields of livestock industry and assisted reproductive technology. The present invention relates specifically to an oocyte maturation promoter and use thereof.

### BACKGROUND ART

In the fields of livestock industry and assisted reproductive technology, a technology has been used in which a sperm and an oocyte are fertilized in vitro (in vitro fertilization), and the resulting fertilized egg (embryo) is transferred to the uterus of another female animal. The quality and condition of the oocyte affect the quality of the fertilized egg, and are thus important factors that influence the transfer result.

In particular, maturation of the oocyte is a prerequisite for successful in vitro fertilization, because an immature oocyte cannot be fertilized with a sperm. However, since the condition of an oocyte cannot be revealed by ultrasound or hormone level, and can be revealed only after oocyte collection, fertilization failure due to poor oocyte maturation is a case often encountered during in vitro fertilization. Therefore, as one of methods for increasing the pregnancy success rate, a method has been sought for maturing a collected oocyte.

Meanwhile, the present inventors have conducted research under the hypothesis that adipose tissue-derived stem cells play a role of feeder cells and also act actively on the reproductive process, and have found that adipose tissue-derived stem cells or a disrupted material thereof exerts an action of activating sperms and oocytes in vitro. Then, patent applications have been filed (see Patent Literatures 1 to 3). For example, Patent Literature 1 describes a method of activating sperm by co-culturing sperm with cells such as adipose tissue-derived stem cells and/or bone marrow-derived stem cells. For example, Patent Literature 2 describes that an oocyte is activated by co-culturing the oocyte with adipose tissue-derived stem cells. In addition, for example, Patent Literature 3 describes a sperm activator comprising, as an active ingredient, a disrupted material from, for instance, adipose tissue-derived stem cells.

### CITATIONS LIST

### Patent Literature

Patent Literature 1: JP 2015-82987 A
Patent Literature 2: JP 2016-7161 A
Patent Literature 3: WO 2018/038180 A

### SUMMARY OF INVENTION

### Technical Problems

In the fields of livestock industry and assisted reproductive technology, the conception efficiency and the pregnancy success rate should be increased.

For example, in the field of livestock industry such as cattle, the production of a large number of child animals having excellent genetic traits leads to an increase in added value of livestock industry products and also improves profitability in livestock industry management. In non-cattle livestock industry, for example in pigs, the number of in vitro fertilization cases has increased in recent years, but is still small as compared to the number of the cow examples. In pigs, polysperm fertilization, in which two or more sperms are fused with the oocyte cytoplasm, frequently occurs. Thus, this does not cause the rate of single fertilization, which is normal fertilization, to increase. In addition, improvement of the success rate of in vitro fertilization in humans is expected to be one means of stopping the extremely low birth rate and aging in this country. As described above, in the fields of livestock industry and assisted reproductive technology, methods have been investigated for improving the conception rate and the fertilization rate.

Meanwhile, in the inventions described in Patent Literatures 1 and 2, sperm and an oocyte are activated by co-culturing the sperm and oocyte with adipose tissue-derived stem cells. However, both use the cells. Therefore, it is necessary to start cell culture at precise timing of their use, so that the adjustment and operation are inconvenient. Besides, there is also a concern about occurrence of adverse events due to use of living cells. The invention described in Patent Literature 3 discloses activation of sperm by using a disrupted material of adipose tissue-derived stem cells, but is silent on oocytes.

### Solutions to Problems

In order to solve the above problems, the present inventors have repeatedly conducted studies focusing on a filtrate of disrupted adipose tissue-derived stem cell solution, and as a result, have found that the filtrate exerts an effect of promoting oocyte maturation in vitro. Then, the present invention has been completed. Specifically, the present invention has the following configuration.
[1] An oocyte maturation promoter comprising, as an active ingredient, a filtrate of disrupted adipose tissue-derived stem cell solution.
[2] The oocyte maturation promoter according to [1], which is used for in vitro fertilization.
[3] The oocyte maturation promoter according to [1] or [2], wherein a biological species of the adipose tissue-derived stem cells and a biological species of the oocyte are identical.
[4] The oocyte maturation promoter according to [1], wherein a biological species of the adipose tissue-derived stem cells is a non-human mammal.
[5] The oocyte maturation promoter according to [1], wherein a biological species of the adipose tissue-derived stem cells is a human.
[6] The oocyte maturation promoter according to [1], wherein a level of ROS is less than 1 when the level in a control is set to 1.
[7] A method for producing an oocyte maturation promoter, comprising the steps of:
(1) disrupting adipose tissue-derived stem cells;
(2) filtering a disrupted cell solution obtained in step (1) or a supernatant obtained by centrifuging the disrupted cell solution to prepare a filtrate; and
(3) providing the filtrate obtained in step (2) as a preparation.
[8] A method for producing a mature oocyte, comprising culturing an oocyte in presence of the oocyte maturation promoter according to [1].
[9] An in vitro fertilization method comprising causing an oocyte treated with the oocyte maturation promoter according to [1] to coexist with a sperm in vitro.
[10] An in vitro fertilization method comprising causing an oocyte and a sperm to coexist in vitro in presence of the oocyte maturation promoter according to [1].
[11] An embryo transfer method comprising: culturing a fertilized embryo produced by the in vitro fertilization method according to [8] or [9]; and injecting the resulting blastocyst into a uterus of a mammal.
[12] Use of the oocyte maturation promoter according to [1] as an oocyte quality enhancer.

### Advantageous Effects of Invention

The oocyte maturation promoter of the present invention can mature an oocyte. More specifically, in in vitro fertilization, each collected oocyte is used, but the oocyte collected by oocyte collection is an immature oocyte. Thus, they cannot be fertilized even if inseminated with sperm. The oocyte maturation promoter of the present invention may be used to mature an immature oocyte. This may facilitate the fertilization process. Since the fertilization rate is improved, and the quality of the fertilized egg and the ability to develop a blastocyst are promoted, the implantation rate and eventually the pregnancy rate are increased. Note that as used herein, the term “maturation” refers to nuclear maturation and cytoplasmic maturation of an oocyte.

In addition, since the oocyte maturation promoter of the present invention uses a filtrate, adverse events due to cell administration can be avoided. For example, generally, in the case of cell administration, the cells to be administered are alive. Hence, if any pathogens are mixed, their inactivation or removal hardly occurs, causing an infection risk.

Further, in the case of cell administration, the cells need to be cultured according to the patient's schedule. In general, freeze-thawed cells are not used for treatment. This is because the activity of the cells may decrease or the cells may die. On the other hand, since the oocyte maturation promoter of the present invention is a cell-free liquid, it can be stored by cryopreservation, and can be thawed, when needed, to be used for treatment. Therefore, it is not necessary to culture the cells at precise timing of their use or to increase the number of cells to the number necessary for treatment. This makes preparation and handling easy, so that clinical advantages are extremely large.

### BRIEF DESCRIPTION OF DRAWINGS

FIG. 1 shows (A) the area of cumulus-oocyte complexes (COCs) before maturation culture and (B) the area of COCs after culture in a test of comparing the areas of cumulus-oocyte complexes.

FIG. 2 shows how mitochondria are distributed in an oocyte (A: Type I in which mitochondria are uniformly distributed throughout cytoplasm; B: Type II in which mitochondria are distributed around the cell membrane and ununiformly distributed inside the cell; and C: Type III in which mitochondria are distributed only around the cell membrane).

### DESCRIPTION OF EMBODIMENTS

The present invention pertains to an oocyte maturation promoter. More specifically, a filtrate of disrupted adipose tissue-derived stem cell solution is contained as an active ingredient. Note that as used herein, the wording “containing, as an active ingredient” means that a filtrate of disrupted adipose tissue-derived stem cell solution is contained in an amount effective for treatment.

### <Oocyte Maturation Promoter>

Note that as used herein, the term “mature oocyte” refers to an oocyte with mature nucleus and mature cytoplasm. The mature oocyte can be fertilized with a sperm and can grow to a blastocyst after fertilization. Note that an immature oocyte refers to an oocyte that cannot be fertilized even if inseminated with sperm.

The oocyte maturation promoter of the present invention may be used to promote oocyte maturation. This likely results in a mature oocyte. Since a mature oocyte can be fertilized with a sperm, the fertilization rate can be improved, and furthermore, the development after fertilization is favorably affected. As a result, the implantation rate and the pregnancy rate are improved.

In the present invention, whether an oocyte is mature or not can be determined such that the oocyte is “mature” if promotion of oocyte maturation can be confirmed by at least one of the following methods. Among them, reactive oxygen species (ROS) also involve the oocyte aging, and thus can be said to be an index of the pregnancy rate particularly in elderly patients.

### (1) to Compare Area of Cumulus-Oocyte Complexes (COCs)

In fertilization, the cumulus cell layer is an important factor. In general, when an oocyte is immature, the cumulus is also small, and as the oocyte matures, the cumulus grows and expands to become larger. The degree of cumulus expansion is considered as an index of oocyte cytoplasmic maturation. Therefore, the area of COCs is measured, and it is herein determined that the oocyte is “mature” when the area is larger than that of the control.

A specific test protocol is shown. Prepare each maturation medium drop (10 μL) containing the oocyte maturation promoter of the present invention. Place an immature oocyte one by one in each drop, photograph COCs with a digital camera before the start of maturation culture, and then culture them in a CO₂ incubator (at 37 to 40° C. and 5% CO₂; the same applies to the following). Photograph the COCs after culture in the same manner, and compare the area of the COCs between before and after maturation culture. Then, the area of the COCs after the maturation culture is divided by the area of the COCs before the maturation culture to give a value A. When the value A is larger than that of the control, it is determined that the oocyte is “mature”. The A is preferably 1.05 times or larger than that of the control and more preferably 1.10 times or larger. In addition, the A is preferably 2.00 times or less than the control.

The maturation medium is not particularly limited as long as the medium is generally used in oocyte maturation culture. Examples of the medium that can be used include NCSU media such as NCSU37 medium or NCSU23 medium, Medium 199, Dulbecco's Modified Eagle's Medium (D-MEM) (e.g., Nacalai Tesque, Sigma, Gibco), DMEM/F12 (e.g., SIGMA, Gibco), and synthetic oviduct fluid (SOF). Two or more kinds of media may be used in combination. If necessary, the medium is optionally admixed with, for instance, serum, plasma, serum albumin, polyvinyl alcohol (PVA), polyvinylpyrrolidone (PVP), antibiotics, 2-mercaptoethanol, amino acids, vitamins, and/or inorganic salts. For culturing, it is preferable to culture cells while using cultureware such as a culture dish.

The culture conditions only need to be conditions generally used in oocyte maturation culture. For example, an environment at 37° C. to 40° C. and 5% CO₂ is preferable.

The camera used for photographing is not particularly limited as long as the area of the COCs can be appropriately measured, but it is preferable to perform photographing by installing a digital camera on an inverted microscope.

### (2) to Evaluate Reactive Oxygen Species (ROS) and Glutathione (GSH) Contents

It is said that oxidative stress is deeply involved in development from an oocyte to an ovum. Oxidative stress-inducing reactive oxygen is known to be a factor causing various diseases. Oxidative stress due to the accumulation of active oxygen causes aging in particular. Oocyte aging causes formation of elliptical or irregular oocyte, which originally is perfectly circular. This results in poor fertilization. In addition, even if fertilization is achieved, cell division becomes difficult or the growth of the embryo may stop. In particular, the decrease in the pregnancy rate of women in and after the second half of thirties is often due to oocyte aging. Reduced glutathione is also said to protect an oocyte from active oxygen toxicity, and the level is used as an index for cytoplasmic maturation of the oocyte. Thus, the levels of reactive oxygen species (ROS) and reduced glutathione (GSH) are herein measured, and when the following requirements are satisfied, the oocyte is determined to be “mature”.

A specific test protocol is shown. Inject 30 to 40 immature oocytes as a group into 200 μL of maturation medium containing the oocyte maturation promoter of the present invention and culture them. Add, after the maturation culture, 0.1% (w/v) hyaluronidase-containing Hepes-Tyrode-Lactate-Pyruvate-polyvinylalcohol (hereinafter, also referred to as Hepes-TLP-PVA), detach cumulus cells by pipetting, and select an oocyte with confirmed release of the first polar body.

Immerse the oocyte in 500 μL of Hepes-TLP-PVA supplemented with 10 μL of 2′,7′-dichlorofluorescein diacetate and 5 μL of Cell Tracker Blue CMF₂HC and stain the oocyte in a CO₂ incubator for 30 minutes. After washing the stained oocyte, photograph and record the fluorescence image with a fluorescence microscope by using each UV filter at 460 nm for ROS and at 365 nm for GSH. A control is set to “1”. Then, an oocyte is determined to be “mature” when ROS is less than 1 or GSH is greater than 1. ROS is preferably 0.9 or less, more preferably 0.8 or less, and still more preferably 0.7 or less. In addition, ROS is preferably 0.1 or larger, and more preferably 0.2 or larger. GSH is preferably 1.0 or larger, more preferably 1.1 or larger, and still more preferably 1.3 or larger. In addition, GSH is preferably 2.0 or less, and more preferably 1.8 or less. As the culture medium and culture conditions, the conditions when the area of COCs is compared can be employed.

### (3) State of Mitochondria Distribution

Mitochondria are organelles that produce energy in vivo, and are also considered to be involved in oocyte aging. Mitochondrial replication is suppressed after oocyte maturation up to morula. Thus, the quality of mitochondria pre-existing in a mature oocyte is important for early embryo development. Here, the present inventors have considered that evaluating the distribution of mitochondria can be used as an index for predicting cytoplasmic maturation. The mitochondria distribution state is herein evaluated, and when the following requirements are satisfied, the oocyte is determined to be “mature”.

One may consult the method of Sha Wei et al. (2010) “Effect of gonadotropins on oocyte maturation in vitro: an animal model.” Fertil Steril, March 15; 93 (5): 1650-61. Specifically, a kit of Mito Tracker (registered trademark) Red CMXRos (Invitrogen) is used and admixed with 149 μL of Medium 199 (Gibco) containing Amikacin (100 mg titer) (Meiji Seika Pharma) per μL of Stock A prepared according to the instruction. Further, 1850 μL of Medium 199 (Gibco) is admixed to form a plurality of 150-μL microdroplets. Each oocyte is selected by the procedure described above.

After washing the selected oocytes, transfer about 10 oocytes as a group to each microdroplet, and allow the microdroplet to stand in a CO₂ incubator for 30 minutes to stain mitochondria. Then, wash the oocytes, photograph them with a confocal laser scanning microscope while using a Rhodamine filter, and conduct image analysis.

Type I is identified if mitochondria are uniformly distributed throughout cytoplasm; Type II is identified if mitochondria are distributed around the cell membrane and ununiformly distributed inside the cell; and Type III is identified if mitochondria are distributed only around the cell membrane. The ratio of Type I to the number of tested oocytes is calculated. When the ratio of Type I in the control is set to “1” and the above ratio is larger than 1, the oocyte of interest is determined to be “mature”. The above ratio is preferably 1.3 or larger and more preferably 1.5 or larger. In addition, the above ratio is preferably 5 or less.

### (4) to Evaluate Mitochondria Activity

Mitochondrial activity is known to be important for porcine oocyte maturation and quality and subsequent embryonic development. Thus, the present inventors have considered that evaluating the mitochondria activity can be used as an index for predicting cytoplasmic maturation. The mitochondria activity is herein evaluated, and when the following requirements are satisfied, the oocyte is determined to be “mature”

One may consult the method of Lee S K et al. (2014) “The association of mitochondrial potential and copy number with pig oocyte maturation and developmental potential.”, J Reprod Dev. 2014 Apr. 24; 60 (2): 128-35. Specifically, a kit of MitoProbe (registered trademark) JC-1 Assay (Invitrogen) is used and admixed with 1092.5 μL of Medium 199 (Gibco) containing Amikacin (100 mg titer) (Meiji Seika Pharma) per 57.5 μL of Stock A prepared according to the instruction to prepare a plurality of 80-μL microdroplets. Each oocyte is selected by the procedure described above.

After washing the selected oocytes, transfer about 10 oocytes as a group to each microdroplet, and allow the microdroplet to stand in a CO₂ incubator for 50 minutes to stain mitochondria. Next, wash the oocytes, then photograph them with a confocal laser scanning microscope while using a Rhodamine filter and an FITC filter, and conduct image analysis. The membrane potential difference is calculated by dividing the fluorescence intensity of red, which indicates high activity, by the fluorescence intensity of green, which indicates low activity, and is defined as mitochondrial activity. When the value of the control is set to “1” and the mitochondrial activity is higher than that, the oocyte is determined to be “mature”. The activity is preferably 1.01 or larger, and more preferably 1.02 or larger. In addition, the activity is preferably 1.10 or less, and more preferably 1.07 or less.

### <Adipose Tissue-Derived Stem Cells>

Adipose tissue-derived stem cells (e.g., Adipose-derived stem cells may be referred to as ASC, Adipose-derived regeneration cells may be referred to as ADSC, Adipose-derived mesenchymal stem cells may be referred to as AT-MSC or AD-MSC; hereinafter, sometimes simply referred to as “ADSC”), which are mesenchymal stem cells, are a type of somatic stem cells and are stem cells contained in adipose tissue. Adipose tissue-derived stem cells also have self-replication capability and multipotency, and are known to be capable of differentiating into not only fat but also various cells such as bone, cartilage, nerve, muscle, myocardium, blood vessel, hepatocyte, and pancreatic islet cells.

The present inventors have found that not the adipose tissue-derived stem cells themselves but a filtrate of disrupted stem cell solution exerts an effect of promoting oocyte maturation.

The origin of ADSCs used in the oocyte maturation promoter of the present invention, that is, the biological species is not particularly limited. It is good to determine the biological species in consideration of the origin of an oocyte to be used and the use of an oocyte (mature oocyte) obtained by the method of the present invention. As long as the desired effect is exerted, that is, the oocyte is matured, the biological species of ADSC used for the oocyte maturation promoter and the biological species of the oocyte may be different from each other, but are preferably the same. In order to avoid the problem of immune rejection, it is preferable to collect ADSCs from the same individual as the subject (recipient) to which the oocyte maturation promoter is applied, and use the collected ADSCs to prepare an oocyte maturation promoter.

Examples of the origin (biological species) of ADSCs include a human and non-human mammals. Examples of the non-human mammals include cows, pigs, horses, goats, sheep, monkeys, dogs, cats, mice, rats, guinea pigs, and hamsters. Among the non-human mammals, cows, pigs, horses, goats, or sheep are preferable in order to efficiently manage livestock industry. In addition, examples of the origin (biological species) of the oocyte include human, non-human mammals, birds, and fish. Examples of the non-human mammals include the animals described above.

### <how to Prepare Adipose Tissue-Derived Stem Cells>

In the present invention, as long as ADSCs maintain pluripotency, cells obtained by culturing (including subculturing) the somatic stem cells also correspond to ADSCs. Usually, ADSCs are prepared in an “isolated state” as cells constituting a cell population (including cells other than ADSCs derived from adipose tissue) while using adipose tissue separated from a living body as a starting material. Here, the term “isolated state” means a state of being taken out from its original environment (that is, a state including a part of a living body), that is, a state of being present in a state different from an original state of being by human operation.

Note that the preparation of ADSCs in the present invention only needs to be performed according to a conventional procedure. ADSCs are widely used in various applications, so that those skilled in the art can also prepare ADSCs with reference to literatures and books. Cells distributed from a public cell bank, commercially available cells, or the like may be used. Hereinafter, as an example of the method for preparing cells, a method for preparing adipose tissue-derived stem cells (one example) will be described.

ADSCs are prepared through steps such as separation, washing, concentration, and culturing of stem cells from adipose matrix. The ADSC preparation method is not particularly limited. For example, ADSCs can be prepared according to known methods (see, for example, Fraser J K et al. (2006), Fat tissue: an under appreciated source of stem cells for biotechnology. Trends in Biotechnology; April; 24 (4): 150-4. Epub 2006 Feb. 20. Review.; Zuk P A et al. (2002), Human adipose tissue is a source of multipotent stem cells. Molecular Biology of the Cell; December; 13 (12): 4279-95.; Zuk P A et al. (2001), Multilineage cells from human adipose tissue: implications for cell-based therapies. Tissue Engineering; April; 7 (2): 211-28). In addition, a device for preparing ADSCs from adipose tissue (e.g., a Celution (registered trademark) device (Cytori Therapeutics, Inc., San Diego, USA)) is also commercially available, and ADSCs may be prepared using the device. The device may be used to separate a cell population containing ADSCs from adipose tissue (K. Lin. et al. Cytotherapy (2008) Vol. 10, No. 4, 417-426). A specific example of the method for preparing ADSCs will be described below.

(1) to Prepare Cell Population from Adipose Tissue

Adipose tissue is collected from humans and non-human mammals by means of, for instance, excision and aspiration. The non-human mammal only needs to be any animal as long as it is the animal described above, and any pet animal, livestock, or laboratory animal is acceptable. The age and sex of the animal are not particularly limited. In order to avoid the problem of immune rejection, it is preferable to collect adipose tissue (autologous adipose tissue) from the same individual as the subject (recipient) to which the oocyte maturation promoter is applied. However, use of the adipose tissue of the same species of animal (allogeneic) or the adipose tissue of a different species of animal is not prohibited.

Note that, in humans, ADSCs can also be prepared from tissue pieces aspirated by liposuction at the time of cosmetic surgery, or excised adipose tissue contained in tissue resected from a living body at the time of, for instance, surgery. ADSCs are present around large blood vessels and can thus be obtained from excised adipose tissue more than from fat aspirate. On the other hand, when the stem cells are prepared from the fat aspirate, the surgical scar is small, and the burden on the donor is also small.

Examples of the adipose tissue include subcutaneous fat, visceral fat, intramuscular fat, and intermuscular fat. Among them, subcutaneous fat can be collected very easily under local anesthesia, so that the burden on the donor at the time of collection is small, and it can be said to be a preferable cell source. Usually, one kind of adipose tissue is used, but two or more kinds of adipose tissue may be used in combination. In addition, adipose tissue (which need not be from the same species of adipose tissue) collected at a plurality of times may be mixed and used for subsequent operations.

The amount of adipose tissue collected may be determined in consideration of the type of donor and the type of tissue, or the amount of ADSCs required, and is, for example, from about 0.5 to 500 g. However, in consideration of the burden on the donor, the amount collected at a time is preferably from about 10 to 20 g or less. The collected adipose tissue is optionally subjected to removal of blood components attached thereto and fragmentation, and then to the following enzyme treatment. Note that the blood components may be removed by washing the adipose tissue in an appropriate buffer or culture solution.

The enzyme treatment is performed by digesting the adipose tissue with an enzyme such as collagenase, trypsin, and/or dispase. Such enzyme treatment only needs to be performed by a method and conditions known to those skilled in the art (see, for example, R. I. Freshney, Culture of Animal Cells: A Manual of Basic Technique, 4th Edition, A John Wiley & Sones Inc., Publication). The cell population obtained by the above enzyme treatment contains pluripotent stem cells, endothelial cells, stromal cells, blood cells, and/or precursor cells thereof. The type and ratio, for example, of cells constituting the cell population depend on the origin and type of the adipose tissue used.

### (2) to Obtain Sedimented Cell Population (Stromal Vascular Fraction: SVF Fraction)

The cell population is subsequently centrifuged. The sediment obtained by centrifugation is recovered as a sedimented cell population (also herein referred to as “SVF fraction”). The centrifugation conditions vary depending on the type and amount of cells, but are, for example, for 1 to 10 minutes and at 800 to 1,500 rpm. Note that prior to the centrifugation, it is preferable that the cell population after the enzyme treatment is, for instance, filtered to remove enzyme-undigested tissues and others contained therein.

The “SVF fraction” obtained here contains ADSCs. Note that the type and ratio, for example, of cells constituting the SVF fraction depend on the origin and type of the adipose tissue used and the conditions for the enzyme treatment. Meanwhile, WO 2006/006692 A1 also shows the characteristics of SVF fraction.

### (3) to Conduct Selective Culture of Adherent Cells (ADSCs) and to Recover Cells

The SVF fraction includes other cell components (e.g., endothelial cells, stromal cells, blood cells, precursor cells thereof) in addition to ADSCs. Thus, in one embodiment of the present invention, the following selective culture is performed to remove unnecessary cell components from the SVF fraction. Then, the resulting cells are used in the present invention as ADSCs.

First, the SVF fraction is suspended in an appropriate culture medium, and then seeded on a culture dish and cultured overnight. Floating cells (non-adherent cells) are removed by medium change. Thereafter, the culture is continued while appropriately replacing the culture medium (e.g., once every 2 to 4 days). If necessary, subculture is performed. The passage number is not particularly limited, but from the viewpoint of maintaining pluripotency and proliferation capability, it is not preferable to repeat the subculture excessively (it is preferable to keep the passage number up to about 5 passages). Note that as the culture medium, a normal culture medium for animal cell culture may be used. Examples of the culture medium that can be used include Dulbecco's Modified Eagle's Medium (DMEM) (e.g., Nissui Pharmaceutical Co., Ltd.), q-MEM (e.g., Dainippon Pharmaceutical Co., Ltd.), DMEM: Ham's F12 mixed medium (1:1) (e.g., Dainippon Pharmaceutical Co., Ltd.), Ham's F12 medium (e.g., Dainippon Pharmaceutical Co., Ltd.), and MCDB201 medium (e.g., Research Institute for the Functional Peptides Co., Ltd.). It is also possible to use a culture medium containing serum (e.g., fetal bovine serum, human serum, sheep serum) or a serum alternative (e.g., Knockout serum replacement (KSR)). The amount of serum or serum alternative added may be set to, for example, within a range of 5 to 30% (v/v).

The above operation makes the adherent cells selectively survive and expand. Subsequently, the expanded cells are recovered. The recovery operation may be performed according to a conventional procedure, and for example, cells after enzyme treatment (trypsin or dispase treatment) may be easily recovered by detaching the cells with a cell scraper, a pipette, or the like. In addition, when sheet culture is performed using, for instance, a commercially available temperature-sensitive culture dish, it is also possible to recover, without enzyme treatment, the cells as they are in the form of a sheet. By using the cells (ADSCs) recovered in this way, a cell population containing highly purified ADSCs may be prepared.

### (4) Low-Serum Culture (Selective Culture in Low-Serum Medium) and Recovery of Cells

In one embodiment of the present invention, the following low-serum culture is performed instead of the operation of (3) or after the operation of (3). Then, the resulting cells are used in the present invention as ADSCs.

In low-serum culture, the SVF fraction (when this step is performed after (3), the cells recovered in (3) are used) is cultured under low-serum conditions to selectively expand pluripotent stem cells of interest (i.e., ADSCs). The low-serum culture method requires only a small amount of serum. Thus, when ADSCs obtained by the method of the present invention are used for therapeutic purposes, the serum of the subject (patient) can be used. That is, culture using autologous serum becomes possible. Here, the wording “under low-serum conditions” is a condition in which 5% (v/v) or less of serum is contained in a culture medium. Preferably, the cells are cultured in a culture solution containing 2% (v/v) or less of serum. More preferably, the cells are cultured in a culture solution containing 2% (v/v) or less of serum and 1 to 100 ng/ML of fibroblast growth factor-2 (bFGF).

The serum is not limited to fetal bovine serum, and human serum or sheep serum, for instance, may be used. When activated sperm obtained by the method of the present invention is used for treatment of humans, human serum is preferably used, and serum of treatment subject (that is, autologous serum) is more preferably used.

As the culture medium, a normal culture medium for animal cell culture may be used under the condition where the amount of serum contained at the time of use is low. Examples of the culture medium that can be used include Dulbecco's Modified Eagle's Medium (DMEM) (e.g., Nissui Pharmaceutical Co., Ltd.), α-MEM (e.g., Dainippon Pharmaceutical Co., Ltd.), DMEM: Ham's F12 mixed medium (1:1) (e.g., Dainippon Pharmaceutical Co., Ltd.), Ham's F12 medium (e.g., Dainippon Pharmaceutical Co., Ltd.), and MCDB201 medium (e.g., Research Institute for the Functional Peptides Co., Ltd.).

By culturing using the above procedure, ADSCs can be selectively expanded. In addition, since ADSCs expanded under the above culture conditions have high proliferation activity, the number of cells required for the present invention can be easily prepared by subculture. Note that WO 2006/006692 A1 shows characteristics of cells selectively expanded by low-serum culture of the SVF fraction.

Subsequently, the cells selectively expanded by the above-described low serum culture are recovered. The recovery operation only needs to be performed in substantially the same manner as in the case of (3) above. By using the cells (ADSCs) recovered, a cell population containing highly purified ADSCs may be prepared.

In the above procedure, cells expanded by low-serum culture of the SVF fraction are used, but cells expanded by directly culturing a cell population obtained from adipose tissue under low-serum conditions (without performing centrifugation to obtain the SVF fraction) may be used as ADSCs. That is, in one embodiment of the present invention, cells expanded when a cell population obtained from adipose tissue is subjected to low-serum culture are used as ADSCs. In addition, instead of the pluripotent stem cells obtained by selective culture (the above (3) and (4)), the SVF fraction (containing adipose tissue-derived mesenchymal stem cells) may be used as it is. Note that the wording “used as it is” means use in the present invention without undergoing selective culture.

### <Method for Preparing Filtrate of Disrupted Adipose Tissue-Derived Stem Cell Solution>

The oocyte maturation promoter of the present invention contains, as an active ingredient, a filtrate (sometimes referred to as an ADSC filtrate) obtained by filtering a disrupted solution obtained by disrupting ADSCs.

ADSCs may be disrupted using a common cell disruption process. For example, a treatment process using, for instance, freeze-thaw (treatment of freezing and then thawing), ultrasonic waves, a French press, a mortar, a homogenizer, or glass beads can be used. In addition, the cells to be subjected to the disruption process may be not only living cells but also dead cells or damaged cells. Among the above disruption processes, freeze-thawing treatment and ultrasonic treatment are preferable. In particular, freeze-thaw treatment is particularly preferable from the viewpoint of being simple, being capable of avoiding contamination due to a contact between a device and cells, and being hygienic. In the case of disruption by freeze-thawing, preferable conditions described later can be used. In the case of disruption by ultrasonic treatment, it is preferable to perform disrupting treatment using unfrozen cells. However, since the denaturation and aggregation of proteins are often caused by the influence of heat emitted from the device, it is preferable to repeatedly perform the treatment for a short time while cooling the cell suspension in ice. Specifically, it is preferable to repeat disruption for 5 to 15 seconds at an output of 200 to 300 W and pausing for 10 to 30 seconds a plurality of times.

Note that in freeze-thawing, cells swell in a freezing process to form ice crystals, and the ice crystals disrupt the cells to be disrupted at the time of thawing. Therefore, in order to sufficiently lyse the cells, it is preferable to repeatedly perform the above. Specifically, the freeze-thawing treatment is preferably repeated 1 to 5 times. Conditions for freezing in the freeze-thawing treatment are not particularly limited, but for example, the freezing is preferably performed at −20 to −196° C. The conditions for thawing are also not particularly limited. For example, it is possible to adopt, for instance, thawing by standing overnight in a refrigerator at 5° C. or lower, thawing by warm-water treatment (e.g., at 35 to 40° C.), or thawing at room temperature.

The concentration of the cell suspension used for disruption is preferably 1×10⁴ to 1×10⁷ ADSC cells/mL. ADSCs are more preferably at 10×10⁴ to 500×10⁴ cells/mL. The concentration is easy to work with, and a sufficient amount of filtrate can be obtained in one operation.

The oocyte maturation promoter of the present invention is prepared using a filtrate obtained by filtering the disrupted cell solution through a filter. Unwanted components can be removed by filter filtration. In addition, when a filter having an appropriate pore size is used, removal of unwanted components and sterilizing filtration can be performed simultaneously. The material of the filter used for the filtering is not particularly limited, but cellulose acetate, which is less likely to adsorb proteins, or a metal-made filter is preferable. In particular, cellulose acetate is preferable. The filter pore size is preferably from 0.1 to 0.45 μm. More preferred is from 0.15 to 0.3 μm. When sterile filtration is also performed simultaneously, the pore size is preferably 0.2 μm.

Note that the disrupted cell solution may be centrifuged in advance, and the obtained supernatant may then be filtered. The disrupted cell solution may be centrifuged before filtering. In this case, the nuclei, for example, may be removed, and clogging of the filter may be prevented, so that the filtering can be efficiently performed. When centrifuged in advance, the disrupted cell solution is preferably centrifuged at 100 to 1,500×g for 3 to 10 minutes after disrupting ADSCs. The temperature during centrifugation is not particularly limited.

If the filtrate is not used immediately for therapy, it can be cryopreserved until use. It is preferable to preserve the filtrate at −100 to −60° C. In general, when cells are subjected to freeze-thawing repeatedly, the activity of cells tends to decrease or the number of dead cells tends to increase. However, the oocyte maturation promoter of the present invention is a filtrate and is free of stem cells. Therefore, the repeated cryopreservation and thawing do not cause a change in the quality. In the present invention, the filtrate obtained is used as an oocyte maturation promoter.

### <Method for Producing Oocyte Maturation Promoter>

The oocyte maturation promoter in the present invention may be produced by the steps below. The ADSC culture method, the disruption process, the centrifugation process, and the filter-filtering process have been described above.
Step (1) of disrupting adipose tissue-derived stem cells;
step (2) of filtering a disrupted cell solution obtained in step (1) or a supernatant obtained by centrifuging the disrupted cell solution to prepare a filtrate; and
step (3) of providing the filtrate obtained in step (2) as a preparation.

The oocyte maturation promoter of the present invention may also be used in the form of a composition when used for various applications described later. Specifically, fetal serum may be included for the purpose of protecting cells as long as the effectiveness is not lost. Further, the obtained filtrate may be made to include other pharmaceutically acceptable components such as a carrier, an excipient, a disintegrant, a buffer, an emulsifier, a suspending agent, a soothing agent, a stabilizer, a preservative, an antiseptic, and/or physiological saline to be provided as a preparation.

The oocyte maturation promoter of the present invention is prepared by disrupting a cell suspension containing ADSCs at 1×10⁴ to 1×10⁷ cells/mL, or preferably a cell suspension containing ADSCs at 10×10⁴ to 500×10⁴ cells/mL. Although the resulting filtrate is actually free of cells, the filtrate contains a material equivalent to cells at 1×10⁴ to 1×10⁷ cells/mL because the cell suspension at the above concentration is disrupted. The material is preferably equivalent to 10×10⁴ to 500×10⁴ cells/mL. The preferred concentration of the cell suspension varies depending on the animal species. However, the lower limit is preferably 10×10⁴ cells/mL or more, 30×10⁴ cells/mL or more, or 50×10⁴ cells/mL or more. The upper limit of the concentration of the cell suspension is preferably 450×10⁴ cells/mL or less, 400×10⁴ cells/mL or less, 380×10⁴ cells/mL or less, 300×10⁴ cells/mL or less, 250×10⁴ cells/mL or less, or 200×10⁴ cells/mL or less. The range of concentration of the cell suspension may be determined as any combination of these upper and lower limits. Note that in the case of a pig, the concentration is more preferably from 30×10⁴ to 150×10⁴ cells/mL, and still more preferably from 40×10⁴ to 130×10⁴ cells/mL. In the case of a cow, the concentration is more preferably from 60×10⁴ to 450×10⁴ cells/mL, and still more preferably from 70×10⁴ to 400×10⁴ cells/mL.

### <Use of Oocyte Maturation Promoter>

The oocyte maturation promoter of the present invention may be used for in vitro fertilization, treatment and improvement of infertility due to functional deterioration of an oocyte, breeding of livestock, maintenance of breeding and species (e.g., maintenance of endangered species, maintenance of pet pedigree, or crossing), and others.

Since the oocyte maturation promoter of the present invention can reduce the level of ROS, it enhances the effect of mitigating the deterioration in quality due to the aging of the oocyte. Thus, the oocyte maturation promoter of the present invention can be used not only as an agent for maturing an oocyte but also as an agent for improving the quality of an oocyte, and is thus expected to exert an effect of improving the pregnancy rate of a human elderly patient.

Hereinafter, in vitro fertilization will be described in detail among uses of the oocyte maturation promoter of the present invention. Incidentally, the conditions characteristic of the present invention, that is, the conditions other than the use of the oocyte maturation promoter of the present invention, only need to be according to a common method (see, for example, Animal Inseminator Course Text (Animal In Vivo Fertilized Egg Transplantation) (Animal Inseminator Association of Japan)).

### <Method of Promoting Maturation of Oocyte During In Vitro Fertilization>

Examples of the method of promoting maturation of an oocyte include culturing an oocyte in the presence of the oocyte maturation promoter of the present invention. In other words, examples of the method for producing a mature oocyte include culturing an oocyte in the presence of the oocyte maturation promoter of the present invention.

The oocyte to be cultured may be an oocyte collected from an ovary, or may be a cryopreserved oocyte. The biological species of the oocyte and the biological species from which the oocyte maturation promoter is derived are preferably the same. Examples include the human and non-human mammal described above.

For the culture, conditions suitable for oocyte culture may be adopted, but for example, culture in an environment at 37 to 40° C. and 5% CO₂ is preferable. Provided that it is preferable to set the temperature close to the body temperature of the animal in consideration of the animal species of the oocyte. For example, in the case of using an oocyte of livestock such as a pig or a cow, the temperature may be set to a relatively high temperature (at 38 to 39° C.). In the case of a human oocyte, the temperature condition is preferably around 37° C. As the medium, the above-described NCSU medium, Medium 199, or the like can be used. Two or more kinds of media may be used in combination. Components that can be added to the culture medium are also as described above. Cells are typically cultured using cultureware such as a culture dish.

The culture method is not particularly limited as long as an oocyte can be exposed to the oocyte maturation promoter by culturing the oocyte together with the oocyte maturation promoter. For example, the oocyte may be placed, suspended, or immersed in a liquid culture medium containing the oocyte maturation promoter. The order of adding the oocyte maturation promoter and the oocyte is not particularly limited, and the oocyte maturation promoter and the oocyte may be added simultaneously and cultured, or may be added with a certain interval and cultured. Specifically, it is preferable to add an oocyte to a culture medium already containing the oocyte maturation promoter.

It is preferable to add 10 to 70 oocytes to 10 to 50 μL of the oocyte maturation promoter, which is a filtrate obtained by disrupting a cell suspension at a concentration of 10×10⁴ to 500×10⁴ cells/mL.

After administration of the oocyte maturation promoter, the oocyte is preferably cultured for 1 hour to 4 days. If the culture period is too short, the oocyte is not sufficiently matured, and thus 1 to 3 days are more preferable.

When the oocyte maturation is successfully found by any of the methods described above, the oocyte is recovered. The oocyte can then be used for in vitro fertilization, infertility treatment caused by deterioration of oocyte function, mating and breeding of livestock, maintenance of breeding and species, and others.

One embodiment of the in vitro fertilization method using the oocyte maturation promoter of the present invention includes (i) a method of allowing an oocyte treated with the oocyte maturation promoter to coexist with sperm in vitro, or (ii) a method of allowing an oocyte and sperm to coexist in vitro in the presence of the oocyte maturation promoter. Here, the coexistence means that two or more entities are present together, and examples thereof include: (i) a method of mixing an oocyte maturation promoter and an oocyte to form a mature oocyte and adding sperm thereto; and (ii) a method of mixing an oocyte maturation promoter, an oocyte, and sperm.

Examples of the sperm to be used in the in vitro fertilization include sperm obtained, for instance, by sperm collection, intra-testis sperm collection, microscopic epididymis sperm collection, percutaneous epididymis sperm collection, or sperm enrichment washing; sperm obtained, for instance, by pre-culturing the sperm; and sperm obtained, for instance, by thawing the sperm after cryopreservation. The sperm is preferably diluted with, for instance, a medium to prepare 50×10⁴ to 4000×10⁴ sperms/mL. In order to increase the fertilization efficiency, it is preferable to use highly motile sperm.

In principle, the biological species of the oocyte and the sperm are identical. In addition, examples of the biological species of the oocyte or sperm include a human and non-human mammal as described above. Provided that the biological species of the oocyte and the biological species of the sperm may be different as long as the combination allows for fertilization.

The culture conditions for an oocyte and sperm or a mature oocyte and sperm may be in accordance with a common in vitro fertilization procedure.

In in vitro fertilization, use of an oocyte matured by the oocyte maturation promoter of the present invention facilitates fertilization, thereby increasing the fertilization rate. When the fertilized egg develops normally, somatic cell division is repeated. Since the subsequent embryo differentiation is also favorable, the rate of development into a morula or blastocyst is improved. Examples of the blastocyst application include an embryo transfer method in which a grown blastocyst is injected into the uterus of a mammal. This leads to improvement in the implantation rate and pregnancy rate. Note that the embryo transfer is not limited to transfer of a blastocyst.

### EXAMPLES

Hereinafter, the present invention will be described in detail with reference to Examples. The present invention, however, is not at all limited to these Examples. The method of preparing an ADSC filtrate used in the experiments, a model animal, and a measurement method are described below. Note that in the Examples, experiments were performed between the same species of animals.

### <To Prepare ADSC Filtrate (by Freeze-Thawing)>

### (1) Porcine Adipose Tissue-Derived Stem Cells (ADSCs)

About 4 g of umbilical cord subcutaneous fat of a slaughtered pig was cut out, collected, and subcultured by the following procedure. The subcultured ADSCs were dispensed into each cell preservation tube and then cryopreserved at −80° C.

The cryopreserved cells in the tube were thawed in a water bath at 38.5° C. and transferred to a 15-mL centrifuge tube. Next, an appropriate amount of 10% FCS-containing PBS (−) was added and the mixture was centrifuged several times. After the supernatant was removed by suction, the sedimented cells were admixed with 1 mL of selection culture solution (25 mL of Mesen PRO (Gibco) supplemented with 2% penicillin-streptomycin, 500 μL of Growth Supply (Gibco), and 250 μL of 200 mM L-Glutamine (Nacalai Tesque)) to resuspend the cells, and the cells were then seeded in a flask and cultured in a CO₂ incubator (38.5° C., 5% CO₂) until confluent. The confluent adipose stem cells were detached with 0.25% (w/v) trypsin/1 mmol/l EDTA, and recovered in a 15-mL centrifuge tube. Further, the inside of the flask was rinsed with 10% FCS-containing PBS (−), and the recovered cell liquid and the rinse liquid were mixed and centrifuged (at 200 g for 5 minutes). After centrifugation, the supernatant was removed by suction, and 300 μL of PBS (−) was added and resuspended. Thereafter, PBS (−) was added to the cell suspension to adjust the concentration, the mixture was dispensed into a cryotube, and then frozen in a freezer at −25° C. to disrupt the cells.

After freezing overnight or longer, the mixture was thawed at room temperature, and after centrifugation (at 1,000 g for 3 min), the supernatant was sterilized by filtration using a 0.2-μm membrane filter (Advantec). After that, PBS (−) was added to the filtrate to adjust the concentration. In this way, an oocyte maturation promoter of the present invention was thus prepared. The oocyte maturation promoter was stored at −25° C. until use, and thawed at room temperature when used.

### (2) Bovine Adipose Tissue-Derived Stem Cells (ADSCs)

Bovine subcutaneous adipose around the umbilical region of the carcass was collected. Substantially the same procedure as for a pig was used to prepare an oocyte maturation promoter.

### <Collection of Oocyte>

### (1) to Collect Porcine Oocytes

Ovaries were harvested from slaughtered pre-puberty female ternary crossbred pigs (hybrid pig crossbred with Landrace species, Large Yorkshire species, and Duroc species). Each collected ovary was immediately immersed in a 0.85% (w/v) sodium chloride solution (physiological saline) supplemented with 100 μg/mL kanamycin sulfate (Meiji Seika Pharma Co., Ltd.) in each 50-mL centrifuge tube. The centrifuge tube was kept at 33 to 36° C., the ovary was washed with physiological saline, and then COCs were aspirated and collected together with follicular fluid from follicles having a diameter of about 1 to 5 mm on the surface of the ovary. The collected COCs were washed with Hepes-TLP-PVA, and only morphologically normal COCs to which cumulus cells were sufficiently attached were selected under a stereomicroscope (OLYMPUS) and subjected to an experiment.

### (2) to Collect Bovine Oocytes

Ovaries were harvested from slaughtered 29 to 30-month-old fattening cows (Japanese Black). Substantially the same procedure as in the porcine oocyte collection was conducted except that COCs were aspirated and collected together with follicular fluid from follicles having a diameter of about 2 to 8 mm on the surface of the ovary.

### <To Prepare Sperm>

### (1) to Prepare Porcine Sperm

Artificial insemination-use semen of the Large White boar (National Livestock Breeding Center, Ibaraki Farm) was used. The artificial insemination-use semen was collected 3 days before insemination. To 1 mL of the artificial insemination-use semen was added 9 mL of sperm washing solution (NaCl: 9 μg/mL, BSA: 1 μg/m, Kanamycin: 0.1 μL), and the mixture was centrifugally washed (at 1, 800 rpm for 5 minutes). After completion of the centrifugal washing, the supernatant was removed, and an in vitro fertilization medium (PFM, Research Institute for the Functional Peptides, Co., Ltd.) warmed to 38.5° C. in advance was added and diluted to prepare a sperm suspension so that the sperm concentration was 1.0×10⁶ sperms/mL.

### (2) To Prepare Bovine Sperm

Artificial insemination-use frozen straw semen of Japanese Black (provided by GIFU Prefectural Livestock Research Institute) was used. Bovine frozen semen filled in the straw and stored in liquid nitrogen was thawed by immersing the straw in warm water at 38° C. for 20 seconds, and washed and centrifuged (at 1, 800 rpm for 5 minutes) twice using BO-Theophylline (BO-medium (Brackett et al., 1975); 10 mM Theophylline) in an equilibrated gas phase in a CO₂ incubator. After centrifugation, the sperm was diluted with BO-Theophylline and BO-BSA-Heparin (BO-medium, 1% Bovine serum albumin, 2.5 IU/ml Heparin), so that the sperm concentration at the time of insemination was 2.0×10⁷ sperms/ml, so as to prepare a sperm suspension.

The sperm vitality of the prepared sperm suspension was checked under an inverted microscope (OLYMPUS). A sperm with the most active forward motion was indicated by +++; a sperm with an active forward motion was indicated by ++; a sperm with a slow forward motion was indicated by +; a sperm with a pivoting or pendulum motion was indicated by +; and a sperm without any motion was indicated by −. The percentage of each sperm in the whole was substituted into the equation below. In order to appropriately determine the effect of the oocyte maturation promoter of the present invention, highly motile sperm, specifically, a sperm suspension having sperm vitality of 70 or more was used in the following experiments.

$$Sperm vitality = \frac{100 ( ++ + ) + 75 ( ++ ) + 50 ( ++ ) + 25 ( \pm ) + 0 ( - )}{100} \qquad [ Formula 1 ]$$

### <In Vitro Maturation Medium>

In the case of a pig, 20 μL of the oocyte maturation promoter of the present invention at various concentrations was each added to 180 μL of microdroplet of an in vitro maturation medium (Medium 199 supplemented with 1 g/mL PVA, 10 IU/mL eCG, 10 I U/mL hCG, 0.01 μg/mL EGF, and 10% pig follicular fluid). Each microdroplet containing 200 μL of in vitro maturation medium served as a control.

In the case of a cow, the same procedure as for a pig was repeated except that an in vitro maturation medium (10% fetal bovine serum-containing Medium 199) was used.

### (1) Test for Comparing Area of Cumulus-Oocyte Complexes (COCs)

Ten drops (each 10 μL) for each in vitro maturation medium drop containing the oocyte maturation promoter of the present invention at a varied concentration were prepared; one immature oocyte was placed in each drop; all COCs were photographed with a digital camera (Canon) placed on an inverted microscope (OLYMPUS) before the start of maturation culture; the COCs were then cultured in a CO₂ incubator (at 38.5° C. and 5% CO₂) (Pig: 42 hours; cow: 22 hours); and thereafter, the COCs in each addition section were photographed in the same manner. The photographed images were analyzed using image analysis software Image J (National Institutes of Health) to measure, average, and compare the area of COCs in each addition section between before and after maturation culture (n=8). Analysis was performed using a one-way analysis of variance test method, and the Tukey-Kramer method was used for comparison between respective addition sections.

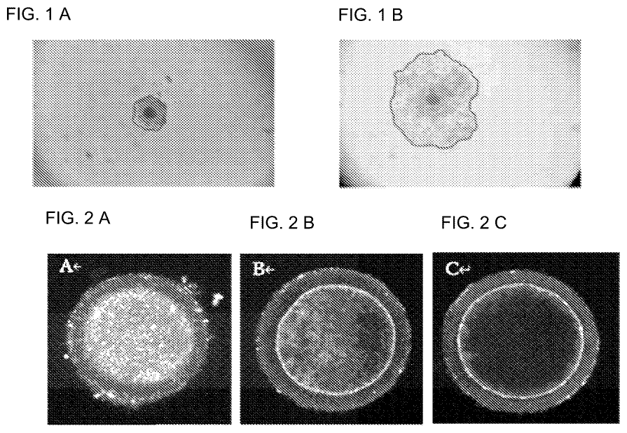

The results of the area comparison test are shown in FIG. 1, Table 1A (pig), and Table 1B (cow). Note that X in Table 1 is obtained by dividing the area of COCs after maturation culture by the area of COCs before maturation culture.

TABLE 1A

| | | | | X: Fold of | |
|---|---|---|---|---|---|
| | | | | increase | |
| Oocyte | | Area of COCs | | in area | |
| maturation | | (mean ± SEM) | | between | |
| promoter | | Before | After | before and | |
| addition | Number of | maturation | maturation | after | Ratio |
| concentration | oocytes | culture | culture | maturation | of X to |
| (cells/mL) | tested | (mm²) | (mm²) | culture | control |
| Control | 80 | 0.53 ± | 1.07 ± | 2.02 | 1.00 |
| | | 0.01 | 0.04 ^{a} | | |
| 50 × 10⁴ | 80 | 0.57 ± | 1.28 ± | 2.25 | 1.11 |
| | | 0.01 | 0.04^{b} | | |
| 100 × 10⁴ | 80 | 0.55 ± | 1.35 ± | 2.45 | 1.22 |
| | | 0.01 | 0.07^{b} | | |
| 200 × 10⁴ | 80 | 0.53 ± | 1.04 ± | 1.96 | 0.97 |
| | | 0.01 | 0.05 ^{a} | | |

^{a} VS.
^{b}P < 0.05

TABLE 1B

| | | | | X: Fold of | |
|---|---|---|---|---|---|
| | | | | increase | |
| Oocyte | | Area of COCs | | in area | |
| maturation | | (mean ± SEM) | | between | |
| promoter | Number | Before | After | before and | |
| addition | of | maturation | maturation | after | Ratio of |
| concentration | oocytes | culture | culture | maturation | X to |
| (cells/mL) | tested | (mm²) | (mm²) | culture | control |
| Control | 52 | 0.7394 ± | 1.1230 ± | 1.52 | 1.00 |
| | | 0.0380 | 0.0882^{a} | | |
| 100 × 10⁴ | 52 | 0.7683 ± | 1.5381 ± | 2.00 | 1.32 |
| | | 0.0383 | 0.0796^{b} | | |
| 200 × 10⁴ | 52 | 0.7783 ± | 1.6624 ± | 2.14 | 1.41 |
| | | 0.0417 | 0.1487^{b} | | |
| 400 × 10⁴ | 52 | 0.7844 ± | 1.4386 ± | 1.83 | 1.21 |
| | | 0.0516 | 0.1029^{ab} | | |

^{a-b}a significant difference was found between different signs (P < 0.05)

FIG. 1 is photographs when 100×10⁴ cells/mL of the pig-derived oocyte maturation promoter of the present invention was added. A is COCs before in vitro maturation culture, and B is COCs after in vitro maturation culture. Table 1 shows the results of quantitatively measuring the area of COCs in each addition section. Tables 1A and 1B have demonstrated that the area of COCs was increased and the cumulus was expanded by the addition of the oocyte maturation promoter of the present invention. In pigs, the area significantly increased depending on the concentration particularly in the sections of 50×10⁴ and 100×10⁴ cells/mL. In cows, particularly in the sections of 100×10⁴ cells/mL and 200×10⁴ cells/mL, the values were found to be significantly higher than that of the control.

Cumulus expansion is considered as an indicator of cytoplasmic maturation, and is said to be involved in oocyte maturation, sperm penetration, and fertilization. It has also been revealed that marked cumulus expansion positively affects the maturation and subsequent embryogenesis potential of porcine and bovine oocytes. Thus, it is believed that cumulus expansion plays a very important role in oocyte maturation and subsequent embryogenesis potential. The above results that the addition of the oocyte maturation promoter of the present invention caused marked cumulus expansion have demonstrated that the oocyte maturation promoter of the present invention contributed to the improvement in oocyte maturation.

### <To Measure Levels of Reactive Oxygen Species (ROS) and Reduced Glutathione (GSH)>

First, 30 to 40 immature oocytes were added as a group to 200 μL of an in vitro maturation medium containing the oocyte maturation promoter of the present invention at a varied concentration, and the mixture was cultured for 42 hours in a CO₂ incubator (at 38.5° C. and 5% CO₂).

After in vitro maturation culture, 0.1% (w/v) hyaluronidase (Sigma-Aldrich)-containing Hepes-TLP-PVA was added, and mechanical pipetting was performed to detach all cumulus cells. Thereafter, an oocyte in which release of the first polar body was found was selected. The oocyte was immersed in 500 μL of Hepes-TLP-PVA supplemented with 10 μL of 2′,7′-dichlorofluorescein diacetate (Sigma-Aldrich) and 5 μL of Cell Tracker Blue CMF₂HC (Invitrogen) and stained in a CO₂ incubator for 30 minutes. The stained oocyte was washed with Hepes-TLP-PVA, and a fluorescence image was then photographed and recorded with a fluorescence microscope (OLYMPUS) equipped with a digital camera (Canon) while using each UV filter at 460 nm for ROS and at 365 nm for GSH. The obtained fluorescence image was converted to a black-white image and then analyzed by image analysis software Image J (National Institutes of Health), and the levels of ROS and GSH contained in each addition section were relatively calculated from the obtained fluorescence luminance with reference to the control (n=3). Analysis was performed using a one-way analysis of variance test method, and the Tukey-Kramer method was used for comparison between respective addition sections.

The results for pigs are shown in Table 2A, and the results for cows are shown in Table 2B.

TABLE 2A

| Oocyte maturation | | | |
|---|---|---|---|
| promoter addition | Number of | | |
| concentration | oocytes | | |
| (cells/mL) | tested | ROS | GSH |
| Control | 30 | 1^{a} | 1^{a} |
| 50 × 10⁴ | 30 | 0.62 ± 0.05^{b} | 1.47 ± 0.10^{b} |
| 100 × 10⁴ | 30 | 0.38 ± 0.01^{b} | 1.62 ± 0.14^{b} |
| 200 × 10⁴ | 30 | 0.92 ± 0.03^{a} | 0.83 ± 0.06^{a} |

^{a}VS.
^{b}P < 0.05

TABLE 2B

| Oocyte maturation | | | |
|---|---|---|---|
| promoter addition | Number of | | |
| concentration | oocytes | | |
| (cells/mL) | tested | ROS | GSH |
| Control | 27 | 1.00 ± 0.04^{a} | 1.00 ± 0.03 |
| 100 × 10⁴ | 29 | 0.85 ± 0.02^{ab} | 1.22 ± 0.05 |
| 200 × 10⁴ | 27 | 0.76 ± 0.04^{b} | 1.15 ± 0.14 |
| 400 × 10⁴ | 28 | 0.94 ± 0.08^{ab} | 1.03 ± 0.10 |

^{a-b}a significant difference was found between different signs (P < 0.05)

Tables 2A and 2B have revealed that the addition of the oocyte maturation promoter of the present invention decreases the level of reactive oxygen species (ROS) and increases the level of glutathione (GSH). In particular, in pigs, the ROS content significantly decreased in a concentration-dependent manner and the GSH content significantly increased in a concentration-dependent manner in the 50×10⁴ and 100×10⁴ cells/mL sections. In cows, in the sections of 100×10⁴ cells/mL and 200×10⁴ cells/mL, the ROS content was found to be significantly low in a concentration-dependent manner. In addition, the GSH content was higher in the sections of 100×10⁴ cells/mL, 200×10⁴ cells/mL, and 400×10⁴ cells/mL than in the control. This has demonstrated that the oocyte maturation promoter of the present invention suppresses oxidative stress and improves the degree of oocyte cytoplasm maturation. In particular, the ROS content decreased, indicating suppression and mitigation of the deterioration in oocyte quality due to oxidative stress.

### <State of Mitochondria Distribution in Mature Oocyte>

The method described in Sha Wei et al. (2010) described above was used as a reference. Porcine oocytes were selected by the procedure described above.

Stock A was prepared according to the instructions by using a kit of Mito Tracker (registered trademark) Red CMXRos (Invitrogen). Here, 1 μL of Stock A was admixed with 149 μL of Medium 199 (Gibco) supplemented with Amikacin (100 mg titer) (Meiji Seika Pharma). Further, 1850 μL of Medium 199 (Gibco) was admixed to form a plurality of 150-μL microdroplets.

Porcine oocytes selected from each addition section were washed with 0.3% (w/v) PVA-PBS, and were transferred, as about 10 oocytes as a group, to each microdroplet. Thereafter, the microdroplet was allowed to stand in a CO₂ incubator for 30 minutes to stain mitochondria. The mature oocyte was washed with 0.3% (w/v) PVA-PBS, photographed under a confocal laser scanning microscope (Carl Zeiss, Oberkochen) using a Rhodamine filter, and then analyzed by image analysis software (Carl Zeiss; ZEN) (n=4). Analysis was performed using a one-way analysis of variance test method; and for the comparison between respective addition sections, a multiple comparison test was performed using the Tukey-Kramer method.

The results are shown in FIG. 2, Table 3A, and Table 3B.

Type I was identified when mitochondria were uniformly distributed throughout cytoplasm (FIG. 2A);

Type II was identified when mitochondria were distributed around the cell membrane and ununiformly distributed inside the cell (FIG. 2B); and

Type III was identified when mitochondria were distributed only around the cell membrane (FIG. 2C).

The distribution of mitochondria was evaluated by calculating each percentage.

TABLE 3A

| Oocyte | | | | |
|---|---|---|---|---|
| maturation | | | | |
| promoter | Number | | | |
| addition | of | | | |
| concentration | oocytes | Type I | Type II | Type III |
| (cells/mL) | tested | (% ± SEM) | (% ± SEM) | (% ± SEM) |
| Control | 37 | 5 | 22 | 10 |
| | | (13.5 ± 2.1) | (59.5 ± 6.0) | (27.0 ± 5.6) |
| 50 × 10⁴ | 41 | 9 | 27 | 5 |
| | | (22.0 ± 4.4) | (65.9 ± 2.9) | (12.2 ± 2.1) |
| 100 × 10⁴ | 31 | 9 | 18 | 4 |
| | | (29.0 ± 4.2) | (58.1 ± 5.0) | (12.9 ± 6.1) |
| 200 × 10⁴ | 33 | 4 | 18 | 11 |
| | | (12.1 ± 4.5) | (54.5 ± 10.6) | (33.3 ± 12.9) |

TABLE 3B

| | Percentage of | |
|---|---|---|
| Oocyte maturation | Type I with | |
| promoter addition | respect to the | |
| concentration | number of | Ratio with respect |
| (cells/mL) | oocytes tested | to control |
| Control | 0.14 | 1 |
| 50 × 10⁴ | 0.22 | 1.62 |
| 100 × 10⁴ | 0.29 | 2.15 |
| 200 × 10⁴ | 0.12 | 0.90 |

Table 3A has demonstrated that the percentage of Type I was higher than that of the control particularly in the sections of 50×10⁴ and 100×10⁴ cells/mL. From Table 3B, particularly in the sections of 50×10⁴ and 100×10⁴ cells/mL, the percentage of Type I with respect to the number of oocytes tested was larger than that of the control, indicating oocyte maturation.

In porcine oocytes that are immature eggs, mitochondria are mainly distributed near the cell membrane, and are then uniformly distributed and spread throughout the cytoplasm as nuclear maturation and cytoplasmic maturation progress. In addition, it has been reported that uniform distribution of mitochondria improves fertilization of an oocyte and development potential of an early embryo. Therefore, it is considered that the addition of the oocyte maturation promoter of the present invention improved the distribution of mitochondria as each maturation and cytoplasmic maturation progressed.

### <To Evaluate Mitochondria Activity>

The method disclosed in Lee S K et al. (2014) described above was used as a reference. Porcine oocytes were selected by the procedure described above.

Stock A was prepared according to the instructions by using a kit of MitoProbe (registered trademark) JC-1 Assay Kit (Invitrogen). Here, 57.5 μL of Stock A was admixed with 1092.5 μL of Medium 199 (Gibco) supplemented with Amikacin (100 mg titer) (Meiji Seika Pharma) to prepare a plurality of 80-μL microdroplets.

Porcine oocytes selected from each addition section were washed with 0.3% (w/v) PVA-PBS, and were transferred, as about 10 oocytes as a group, to each microdroplet. Thereafter, the microdroplet was allowed to stand in a CO₂ incubator for 50 minutes to stain mitochondria. After staining, each mature oocyte was immediately washed with 0.3% (w/v) PVA-PBS, photographed on a confocal laser scanning microscope (Carl Zeiss) using a Rhodamine filter and an FITC filter, and then analyzed by image analysis software (Carl Zeiss; ZEN).

The fluorescence intensity of the image was measured using Image J software. The membrane potential difference was calculated by dividing the fluorescence intensity of red, which indicates high activity, by the fluorescence intensity of green, which indicates low activity, to evaluate the mitochondrial activity (n=3). Analysis was performed using a one-way analysis of variance test method, and the Tukey-Kramer method was used for comparison between respective addition sections.

The results are shown in Table 4. Table 4 shows the membrane potential difference of each addition section relative to the control.

TABLE 4

| Oocyte maturation | | |
|---|---|---|
| promoter addition | Number of | |
| concentration | oocytes | Mitochondrial activity |
| (cells/mL) | tested | (red/green) |
| Control | 18 | 1.000 ± 0.0057^{b} |
| 50 × 10⁴ | 26 | 1.020 ± 0.0089^{ab} |
| 100 × 10⁴ | 21 | 1.037 ± 0.0057^{a} |
| 200 × 10⁴ | 21 | 1.007 ± 0.0078^{ab} |

^{a-b}a significant difference was found between different signs (P < 0.05)

The results in Table 4 have demonstrated that the addition of the oocyte maturation promoter of the present invention significantly increased the mitochondrial activity as compared with the control, particularly in the section of 100×10⁴ cells/mL. Mitochondrial activity is known to be important for porcine oocyte maturation and quality and subsequent embryonic development. The results in Table 4 have revealed that the oocyte maturation promoter of the present invention improves the mitochondrial function. It is also known that low mitochondrial activity causes apoptosis. Thus, the improvement in mitochondrial activity by the oocyte maturation promoter of the present invention also leads to suppression of early apoptosis. In this way, the oocyte cytoplasmic maturation is induced by the oocyte maturation promoter of the present invention. It has been presumed that this would sufficiently maintain the subsequent embryonic development capability, and thus contribute to improvement of the fertilization rate, implantation rate, and pregnancy rate.

### <In Vitro Fertilization>

In order to check whether or not an oocyte using the oocyte maturation promoter of the present invention was normally subjected to in vitro fertilization, COCs were subjected to in vitro maturation culture in an in vitro maturation medium containing the oocyte maturation promoter of the present invention at various concentrations, and in vitro fertilization was then performed.

First, several dozens of immature oocytes (pig: 30 to 40 oocytes; cow: 20 to 35 oocytes) were added as a group to 200 μL of an in vitro maturation medium containing 20 μL of the oocyte maturation promoter of the present invention at a varied concentration, and the mixture was cultured in a CO₂ incubator (at 38.5° C. and 5% CO₂) (pig: 42 hours; cow: 22 hours). Next, 0.1% (w/v) hyaluronidase (Sigma-Aldrich)-containing Hepes-TLP-PVA was sprinkled on the COCs, and the remaining cumulus cells were detached using a glass pipette while leaving about 2 or 3 layers. Then, the cells were washed multiple times with 80 μL of microdroplets of an in vitro fertilization-use culture medium (PFM, Research Institute for the Functional Peptides, Co., Ltd.), and 20 to 40 oocytes were transferred as a group into 90 to 180 μL of each microdroplet of an in vitro fertilization-use culture medium. Then, 10 to 20 μL of a sperm suspension was added thereto and subjected to insemination. The final sperm concentration was adjusted to 1.0×10⁶ sperms/mL for a pig and 2.0×10⁷ sperms/mL for a cow. Thereafter, the cells were co-cultured for 6 hours after insemination in a CO₂ incubator (at 38.5° C. and 5% CO₂), and the oocytes were then transferred to a development medium and cultured for 6 hours. After that, the in vitro fertilization status, the maturation rate, and the normal fertilization rate were evaluated according to the following protocols, and compared and examined between respective addition sections. Analysis was performed using a one-way analysis of variance test method; and for the comparison between respective addition sections, a multiple comparison test was performed using the Tukey-Kramer method.

### (1) to Check Status of In Vitro Fertilization

After 12 hours from the insemination, some eggs of each addition section were randomly taken out from the development medium, and fixed with a Carnoy's solution (ethanol:acetic acid=3:1) under a stereomicroscope. After 48 hours from the fixation, the nuclei were stained with 1% (w/v) acetic orcein solution, the fertilization status was observed under an upright microscope (OLYMPUS), and judged as follows (n=5):
(i) maturation rate: percentage of eggs, in which the first polar body and chromosomes during the second meiotic metaphase were observed, with respect to the eggs tested;
(ii) sperm penetration rate: percentage of eggs, in which a sperm head or one or more male pronuclei were observed, with respect to mature eggs;
(iii) normal fertilization rate: percentage of eggs, in which the first and second polar bodies were released and both male and female pronuclei were observed, with respect to eggs into which a sperm penetrated;
(iv) polysperm fertilization rate (common in pigs): percentage of eggs, in which three or more pronuclei were observed after release of the first and second polar bodies, with respect to the eggs into which sperm penetrated; and
(v) fertilization efficiency: percentage of normally fertilized eggs with respect to mature eggs.

Table 5A shows the above results in pigs. Table 5B shows the results of the maturation rate in cows, and Table 5C shows the results of the normal fertilization rate.

TABLE 5A

| Oocyte | | | | | | |
|---|---|---|---|---|---|---|
| maturation | | | | | | |
| promoter | | | Details of fertilization status (mean ± SEM) | | | |
| addition | Number of | | Sperm | Normal | Polysperm | |
| concentration | oocytes | Maturation | penetration | fertilization | fertilization | Fertilization |
| (cells/mL) | tested | rate (%) | rate (%) | rate (%) | rate (%) | efficiency (%) |
| Control | 50 | 36 | 28 | 8 | 20 | 8 |
| | | (72.0 ± 3.7) | (77.8 ± 2.7) | (28.6 ± 4.4) ^{a} | (71.4 ± 4.4) ^{a} | (22.2 ± 4.0) ^{a} |
| 50 × 10⁴ | 50 | 39 | 33 | 22 | 11 | 22 |
| | | (78.0 ± 3.7) | (84.6 ± 2.4) | (66.7 ± 1.4) ^{b} | (33.3 ± 1.4) ^{b} | (56.4 ± 1.9) ^{b} |
| 100 × 10⁴ | 50 | 41 | 35 | 28 | 7 | 28 |
| | | (82.0 ± 3.7) | (85.4 ± 3.0) | (80.0 ± 3.0) ^{b} | (20.0 ± 3.0) ^{b} | (68.2 ± 4.9) ^{b} |
| 200 × 10⁴ | 50 | 34 | 28 | 9 | 16 | 9 |
| | | (68.0 ± 2.0) | (77.8 ± 2.7) | (36.0 ± 4.0) ^{a} | (64.0 ± 4.0) ^{a} | (26.4 ± 3.2) ^{a} |

^{a} VS
^{b} P < 0.05

TABLE 5B

| Oocyte maturation | | |
|---|---|---|
| promoter addition | Number of | |
| concentration | oocytes | Maturation rate (%) |
| (cells/mL) | tested | (mean ± SEM) |
| Control | 50 | 31 (62.0 ± 3.4) ^{a} |
| 100 × 10⁴ | 51 | 40 (78.4 ± 3.7) ^{b} |
| 200 × 10⁴ | 46 | 36 (78.3 ± 3.7) ^{b} |
| 400 × 10⁴ | 50 | 38 (76.0 ± 3.9) ^{ab} |

^{a-b} a significant difference was found between different signs (P < 0.05)

TABLE 5C

| Oocyte maturation | | |
|---|---|---|
| promoter addition | | |
| concentration | Number of oocytes | Normal fertilization |
| (cells/mL) | tested | rate (%) (mean ± SEM) |
| Control | 40 | 22 (55.0 ± 4.8) ^{a} |
| 100 × 10⁴ | 41 | 28 (68.3 ± 1.0) ^{bc} |
| 200 × 10⁴ | 36 | 25 (69.4 ± 5.6) ^{b} |
| 400 × 10⁴ | 40 | 24 (60.0 ± 6.0) ^{ac} |

^{a-b-c} a significant difference was found between different signs (P < 0.05)

As evident from Table 5A, the maturation rate, sperm penetration rate, normal fertilization rate, and fertilization efficiency increased in pigs, particularly in the 50×10⁴ and 100×10⁴ cells/mL sections. On the other hand, the polysperm fertilization rate decreased particularly in the 50×10⁴ and 100×10⁴ cells/mL sections. Tables 5B and 5C have clearly demonstrated that in cows, the maturation rate and the normal fertilization rate increased particularly in the sections of 100×10⁴ cells/mL and 200×10⁴ cells/mL, and among them, 200×10⁴ cells/mL resulted in the highest value.

The cumulus cell secretory factor includes an important factor that induces meiotic division in the oocyte, and is said to be involved in the progress of oocyte nuclear maturation during in vitro maturation culture process. Table 1 clearly indicates marked cumulus expansion upon the addition of the oocyte maturation promoter of the present invention. Perhaps, the oocyte maturation promoter of the present invention might cause secretion of many maturation-related factors from cumulus cells, thereby promoting oocyte nuclear maturation. Similarly, it is presumed that cumulus expansion contributed to the increase in the sperm penetration rate. It is also said that the sperm nucleus decondensation and the male pronucleation depend on the oocyte maturation state. Therefore, it can be said that use of an oocyte to which the oocyte maturation promoter for promoting maturation of an oocyte in the present invention is added reasonably increases the fertilization efficiency.

On the other hand, it is said that incomplete cytoplasmic maturation leads to a delay of the polysperm fertilization rejection mechanism, thereby increasing the polysperm fertilization rate. Pigs, in particular, have more polysperm fertilization than other animal species. Since the polysperm fertilized eggs do not normally develop, this is one of the factors that inhibit improvement in the production efficiency of in vitro fertilized embryos of pigs. However, the results of Table 5A have clearly demonstrated that the oocyte maturation promoter of the present invention possibly improves the cytoplasmic maturation status. As a result, it is considered that the polysperm fertilization rejection mechanism normally functions, and thus the normal fertilization rate is improved and the polysperm fertilization rate is decreased.

### (2) to Check Cleavage Rate and Blastocyst Formation Status

Even if normal fertilization is achieved, the egg does not grow into a good blastocyst unless subsequent somatic cell division (cleavage) is appropriately performed. In addition, the cleavage may be appropriately performed. However, the implantation rate is not improved if the egg is returned to the uterus but is not normally developed or differentiated thereafter. Therefore, the following procedure was used to measure the cleavage rate and the subsequent blastocyst formation status when in vitro fertilization was performed using the oocyte maturation promoter of the present invention.

Specifically, in vitro fertilization was performed on pigs and cows by the above method, and the cleavage state was then observed under an inverted microscope (OLYMPUS) two days after insemination, and the cleavage rate, which is the percentage of eggs with confirmed cell division, was calculated. After 7 days from the insemination, the blastocyst formation status was observed, and the blastocyst formation rate, which is the percentage of embryos with a confirmed blastocyst cavity, was calculated. Further, each obtained blastocyst was subjected to Hoechst 33342 staining to measure the total number of blastocyst cells, and TUNEL staining to measure the percentage of apoptotic cells with respect to the total number of blastocyst cells. These numbers were then compared and examined between respective addition sections (n=3). Analysis was performed using a one-way analysis of variance test method; and for the comparison between respective addition sections, a multiple comparison test was performed using the Tukey-Kramer method.

Table 6A shows the results of the cleavage rate and the cleavage status up to the 4-cell stage in pigs; Table 6B shows the formation status from then on to the blastocyst; and Table 6C shows the results of the percentage of apoptotic cells. Table 7A shows the cleavage status and blastocyst formation rate up to the 2-cell stage in cows; and Table 7B shows the results of the total number of blastocyst cells and the percentage of apoptotic cells.

TABLE 6A

| Oocyte maturation | Number | | | | |
|---|---|---|---|---|---|
| promoter addition | of | | | | |
| concentration | oocytes | Cleavage | Details of cleavage status (mean ± SEM) | | |
| (cells/mL) | tested | rate (%) | 2 cell | 3 cell | 4 cell≤ |
| Control | 90 | 52 | 18 | 9(12.2 ± | 25 |
| | | (57.8 ± 1.1) ^{a} | (20.0 ± 6.9) | 2.9) ^{ab} | (25.5 ± 2.9 ^{c} |
| 50 × 10⁴ | 90 | 64 | 12 | 13 | 39 |
| | | (71.1 ± 1.1) ^{b} | (13.3 ± 0.1) | (14.5 ± 2.2) ^{ab} | (43.3 ± 1.9) ^{ab} |
| 100 × 10⁴ | 90 | 71 | 7 | 19 | 45 |
| | | (78.9 ± 1.1) ^{c} | (7.8 ± 2.2) | (21.1 ± 2.9) ^{b} | (50.0 ± 0.1) ^{b} |
| 200 × 10⁴ | 90 | 44 | 12 | 7 | 25 |
| | | (48.9 ± 1.1) ^{d} | (13.3 ± 3.3) | (7.8 ± 1.1) ^{a} | (27.8 ± 2.9) ^{c} |

^{a} VS.
^{b} VS.
^{c} VS.
^{d} P < 0.05

TABLE 6B

| Oocyte | | | | | | | |
|---|---|---|---|---|---|---|---|
| maturation | | | Developmental stage of blastocyst stage embryo | | | | |
| promoter | Number | | (mean ± SEM) | | | | |
| addition | of | Blastocyst | | | | Blastocyst | |
| concentration | oocytes | formation | Early | | Expanded | during | Hatched |
| (cells/mL) | tested | rate (%) | blastocyst | Blastocyst | blastocyst | hatching | blastocyst |
| Control | 90 | 14 (15.6 ± | 6 (6.7 ± | 7 (7.8 ± | 1 (1.1 ± | 0 (0.0 ± | 0 (0.0 ± |
| | | 1.1) ^{a} | 1.9) | 1.1) ^{ab} | 1.1) ^{a} | 0.0) | 0.0) |
| 50 × 10⁴ | 90 | 22 (24.4 ± | 3 (3.3 ± | 11 (12.2 ± | 4 (4.4 ± | 2 (2.2 ± | 1 (1.1 ± |
| | | 1.1) ^{b} | 1.9) | 1.1) ^{b} | 1.1) ^{a} | 1.1) | 1.1) |
| 100 × 10⁴ | 90 | 26 (28.9 ± | 4 (4.5 ± | 8 (8.9 ± | 11 (12.2 ± | 1 (1.1 ± | 2 (2.2 ± |
| | | 2.2) ^{b} | 2.2) | 1.1) ^{ab} | 2.9) ^{b} | 1.1) | 1.1) |
| 200 × 10⁴ | 90 | 10 (12.2 ± | 4 (4.4 ± | 5 (5.53 ± | 1 (1.1 ± | 1 (1.1 ± | 0 (0.0 ± |
| | | 1.1) ^{a} | 1.1) | 2.23) ^{a} | 1.1) ^{a} | 1.1) | 0.0) |

^{a} VS.
^{b} VS.
^{c} P < 0.05

TABLE 6C

| Oocyte maturation | Number | Total number | |
|---|---|---|---|
| promoter addition | of | of blastocyst | Apoptotic |
| concentration | oocytes | cells | Cell Index |
| (cells/mL) | tested | (mean ± SEM) | (% ± SEM) |
| Control | 90 | 44.5 ± 2.6^{a} | 12.1 ± 0.5^{a} |
| 50 × 10⁴ | 90 | 62.3 ± 4.1^{b} | 5.6 ± 0.3^{b} |
| 100 × 10⁴ | 90 | 66.2 ± 5.1^{b} | 4.0 ± 0.3^{b} |
| 200 × 10⁴ | 90 | 48.1 ± 3.9^{ab} | 9.8 ± 1.0^{a} |

^{a}VS.
^{b}VS.
c: P < 0.05

TABLE 7A

| | |
|---|---|
| Oocyte | |
| maturation | Details of cleavage status |
| promoter | (mean ± SEM) |

| addition | Number of | 2 days after | 7 days after |
|---|---|---|---|
| concentration | oocytes | insemination | insemination |
| (cells/mL) | tested | 2 cell ≤ | Blastocyst |
| Control | 101 | 46 (45.5 ± | 19 (18.8 ± |
| | | 3.4) | 2.1) ^{a} |
| 100 × 10⁴ | 95 | 51 (53.7 ± | 22 (23.2 ± |
| | | 4.8) | 3.1) ^{ab} |
| 200 × 10⁴ | 100 | 56 (56.0 ± | 31 (31.0 ± |
| | | 2.2) | 2.6) ^{b} |
| 400 × 10⁴ | 101 | 46 (45.5 ± | 23 (22.8 ± |
| | | 3.9) | 2.9) ^{ab} |

^{a-b}a significant difference was found between different signs (P < 0.05)

TABLE 7B

| Oocyte | | | | |
|---|---|---|---|---|
| maturation | | | | |
| promoter | Number | Total | | Apoptotic |
| addition | of | number of | Number of | Cell |
| concentration | oocytes | blastocyst | apoptotic | Index |
| (cells/mL) | tested | cells | cells | (% ± SEM) |
| Control | 10 | 138.6 ± | 8.3 ± 1.0 | 6.2 ± |
| | | 12.4^{a} | | 0.9^{a} |
| 100 × 10⁴ | 10 | 166.3 ± | 8.2 ± 2.3 | 4.3 ± |
| | | 17.3^{ab} | | 0. 9^{ab} |
| 200 × 10⁴ | 10 | 197 ± | 6.7 ± 1.5 | 3.2 ± |
| | | 11.9^{b} | | 0.5^{b} |
| 400 × 10⁴ | 10 | 154.6 ± | 5.8 ± 2.0 | 3.9 ± |
| | | 11.5^{ab} | | 1.6^{b} |

^{a-b}a significant difference was found between different signs (P < 0.05)

From the results in Table 6A, when in vitro fertilization was performed using a mature egg by adding the oocyte maturation promoter of the present invention, in pigs, the cleavage rate of the fertilized egg significantly increased particularly in the sections of 50×10⁴ and 100×10⁴ cells/mL, and an improvement in the development rate was detected. Generally, an embryo during an early development stage is controlled by factors such as maternal mRNAs and proteins accumulated in the cytoplasm of an egg during the maturation culture process. This result indicates that the addition of the oocyte maturation promoter of the present invention during the maturation culture process improved cytoplasmic maturation, and more maternal factors were accumulated intracellularly, so that the early embryonic development capability was improved.

In addition, the results of Tables 6B and 6C show a significant increase in blastocysts at each developmental stage even in the subsequent development in pigs. This is presumed to be because the cleavage status was good. In addition, a decrease in the apoptotic cell index was also observed.

In cows, as evident from Table 7A, the addition of the oocyte maturation promoter of the present invention improved the cleavage rate in all sections. In particular, the highest value was obtained in the section of 200×10⁴ cells/mL. The blastocyst formation rate also exhibited a significantly higher value in the 200×10⁴ cells/mL section than in the control, and was the highest value among the respective addition sections. Further, morphologically normal blastocysts were obtained in all experimental sections.

Furthermore, Table 7B has clearly indicated a significantly higher value than in the control, and the total number of blastocyst cells was the highest value in the 200×10⁴ cells/mL section among the respective addition sections. The number of apoptotic cells and the percentage of apoptotic cells with respect to the total number of blastocyst cells tended to decrease in all the oocyte maturation promoter addition sections as compared with the control, and a remarkable effect was observed particularly in the sections of 200×10⁴ and 400×10⁴ cells/mL.

### <To Evaluate Quality of Blastocyst>

Table 6C and Table 7B have demonstrated that the addition of the oocyte maturation promoter of the present invention significantly improves the blastocyst total cell count formation rate. However, blastocysts may be graded, so that it is necessary to selectively transfer a high-grade blastocyst, that is, a blastocyst having a high possibility of implantation. Blastocysts may be graded by evaluating, for instance, how large the number of cells in a part to be a fetus (inner cell mass=ICM) and a part to be a placenta (trophectoderm=TE) (Gardner classification).

Here, the following protocol was used to evaluate the differentiation rate of ICM or TE in the blastocyst when the oocyte maturation promoter of the present invention was added.

In the case of pigs, in vitro fertilization was carried out by the above-described method. The blastocyst that was obtained 7 days after insemination and was morphologically recognized as a blastocyst was immersed in 6 μL of 0.5% (w/v) Pronase solution to dissolve a zona pellucida. The blastocyst was washed with Hepes-TLP-PVA, and admixed with a reaction solution A (10 μL of Anti-Pig Serum antibody produced in rabbit (Sigma-Aldrich) and 50 μL of PBS (−)). The mixture was then stained in a CO₂ incubator for 1 hour. Next, the mixture was washed with Hepes-TLP-PVA, and admixed with a reaction solution B (5 μL of complement sera from guinea pig (Sigma-Aldrich) and 50 μL of PI-Hoechst 33342-PBS stock). The mixture was stained again in a CO₂ incubator for 1 hour. After staining, the blastocysts were washed with Hepes-TLP-PVA, and fluorescence images were recorded under a fluorescence microscope (Olympus) equipped with a digital camera while using a 365-nm UV filter. The stained nuclei were counted, and when the number of cells was 32 or more, the cells were identified as a blastocyst, and the total number of blastocyst cells was calculated. In addition, ICM cells stained with blue fluorescence and TE cells stained with red fluorescence were counted, and the ratio of ICM cells and the ratio of TE cells to the total number of blastocyst cells were calculated.

In the case of cows, in vitro fertilization was carried out by the above-described method. Morphologically recognized blastocysts obtained 7 days after insemination were immersed and fixed in 500 μL of 4% (w/v) paraformaldehyde for 30 minutes at room temperature under light-shielding conditions. The fixed embryos were washed several times with 0.3% PVA-PBS, immersed for 30 minutes in 1% (w/v) Triton X-100 (Sigma-Aldrich)-containing PVA-PBS at room temperature under light-shielding conditions for permeabilization. After the permeabilization, the embryos were washed several times with 0.3% PVA-PBS, and immersed in PVA-PBS containing 0.5% BSA and 10% Horse serum (16050-130, Gibco, Thermo Fisher Scientific) for 2 hours at 4° C. under light-shielding conditions to block non-specific binding of the antibody. After blocking, the embryos were immersed, in a primary antibody CDX2 (D11D10) Rabbit mAb (Cell Signaling Technology, Danvers, MA, USA) solution diluted 1:50 with 0.3% PVA-PBS, at 4° C. for 24 hours under light-shielding conditions. Each embryo after the primary antibody reaction was washed several times with 0.3% PVA-PBS and immersed, in a secondary antibody Anti-rabbit IgG (H+L), F (ab′)₂ Fragment (Alexa Fluor (registered trademark) 555 Conjugate; Cell Signaling Technology) solution diluted 1:500 with 0.3% PVA-PBS, at 4° C. for 1 hour under light-shielding conditions to stain TE cells (with red fluorescence). Next, the nuclei were stained by immersion in 10 μg/mL Hoechst 33342-containing 0.3% PBS-PVA for 15 minutes at 4° C. under light-shielding conditions, mounted on a slide glass, and covered with a cover glass while the surrounding was sealed with a topcoat. For the prepared hole-mount specimen, a fluorescence image was recorded and observed under a fluorescence microscope (OLYMPUS) equipped with a digital camera (Canon) while using a UV filter at 365 nm for Hoechst 33342 and a UV filter at 546 nm for Alexa Fluor (registered trademark) 555. From the obtained fluorescence image, nuclei stained with blue fluorescence and nuclei stained with red fluorescence were counted using image analysis software Image J (National Institutes of Health), and the total number of cells and the number of TE cells were measured. In addition, the number of ICM cells was calculated by subtracting the number of TE cells from the total number of cells.

Table 8A shows the results in pigs and Table 8B shows the results in cows. The percentage of ICM cells and the percentage of TE cells indicate the ratio of the number of ICM cells and the number of TE cells to the total number of cells.

TABLE 8A

| Oocyte | | Total | | | | |
|---|---|---|---|---|---|---|
| maturation | | number | Number | | Number | |
| promoter | Number | of | of ICM | Percentage | of TE | Percentage |
| addition | of | cells | cells | of ICM | cells | of TE |
| concentration | oocytes | (count ± | (count ± | cells | (count ± | cells |
| (cells/mL) | tested | SEM) | SEM) | (% ± SEM) | SEM) | (% ± SEM) |
| Control | 13 | 44.5 ± | 4.7 ± | 10.6 ± | 39.8 ± | 89.4 ± |
| | | 2.2^{b} | 0.6^{b} | 1.2^{ab} | 2.0 | 1.2 |
| 50 × 10⁴ | 13 | 53.3 ± | 5.8 ± | 10.9 ± | 47.5 ± | 89.1 ± |
| | | 3.4^{ab} | 0.4^{b} | 0.5^{ab} | 3.1 | 0.5 |
| 100 × 10⁴ | 13 | 60.8 ± | 7.7 ± | 13.1 ± | 53.1 ± | 86.9 ± |
| | | 4.9^{a} | 0.4^{a} | 0.6^{a} | 4.7 | 0.6 |
| 200 × 10⁴ | 13 | 47.1 ± | 4.8 ± | 10.1 ± | 42.3 ± | 89.9 ± |
| | | 3.5^{ab} | 0.4^{b} | 0.5^{ab} | 3.1 | 0.5 |

^{a-b}a significant difference was found between different signs (P < 0.05)

TABLE 8B

| Oocyte | | Total | | | | |
|---|---|---|---|---|---|---|
| maturation | | number | Number | | Number | |
| promoter | Number | of | of ICM | Percentage | of TE | Percentage |
| addition | of | cells | cells | of ICM | cells | of TE |
| concentration | oocytes | (count ± | (count ± | cells | (count ± | cells |
| (cells/mL) | tested | SEM) | SEM) | (% ± SEM) | SEM) | (% ± SEM) |
| Control | 10 | 127.4 ± | 43 ± | 33.3 ± | 84.4 ± | 66.6 ± |
| | | 9.6^{a} | 5.1 | 2.5 | 6.3 | 2.5 |
| 100 × 10⁴ | 10 | 166.2 ± | 63.1 ± | 38.1 ± | 103.2 ± | 61.9 ± |
| | | 11.2^{ab} | 5.8 | 2.4 | 8.4 | 2.4 |
| 200 × 10⁴ | 10 | 171.9 ± | 64.4 ± | 36.7 ± | 106.9 ± | 62.8 ± |
| | | 8.2^{b} | 7.1 | 3.3 | 5.8 | 3.0 |
| 400 × 10⁴ | 10 | 154 ± | 56.1 ± | 37.4 ± | 97.9 ± | 62.6 ± |
| | | 13.4^{ab} | 5.0 | 2.5 | 10.6 | 2.5 |

^{a-b}a significant difference was found between different signs (P < 0.05)

The results in Table 8A have demonstrated that the addition of the oocyte maturation promoter of the present invention increases the number of cells in all cases. In pigs, the total number of cells, the number of ICM cells, and the percentage of ICM cells were significantly increased particularly at concentrations of 50×10⁴ cells/mL and 100×10⁴ cells/mL.

In cows, particularly in the 200×10⁴ cells/mL section, the total number of cells exhibited a significantly higher value than that of the control, and the number of ICM cells and the number of TE cells also each exhibited a higher value than that of the control in all experimental sections. In addition, the results of the ratio of the number of ICM cells to the total number of blastocyst cells have indicated that the rate of differentiation into ICM tended to increase in all experimental sections.

Evaluation of the number of these cells in each blastocyst stage embryo is an indicator of embryo quality. In particular, ICM cells have been found to be involved in implantation after embryo transfer and normal development of the fetus. This has demonstrated that high-quality blastocysts were produced by the addition of the oocyte maturation promoter of the present invention. It is presumed that the effect of improving the implantation rate and the pregnancy rate after implantation is exerted.

### INDUSTRIAL APPLICABILITY

The oocyte maturation promoter of the present invention can promote oocyte maturation, thereby improving the fertilization rate. The present invention is effective for use in mating and breeding of livestock (for improvement in the success rate and efficiency of embryo transfer), maintenance of breeding and species (e.g., maintenance of endangered species, maintenance of pet pedigree, or crossing), and so on. In addition, it is also effective for treatment of infertility caused by functional deterioration such as poor oocyte maturation. Besides, the quality of aged oocyte can also be improved.

1. An oocyte maturation promoter comprising, as an active ingredient, a filtrate of disrupted adipose tissue-derived stem cell solution.

2. The oocyte maturation promoter according to claim 1, which is used for in vitro fertilization.

3. The oocyte maturation promoter according to claim 1, wherein a biological species of the adipose tissue-derived stem cells and a biological species of the oocyte are identical.

4. The oocyte maturation promoter according to claim 1, wherein a biological species of the adipose tissue-derived stem cells is a non-human mammal.

5. The oocyte maturation promoter according to claim 1, wherein a biological species of the adipose tissue-derived stem cells is a human.

6. The oocyte maturation promoter according to claim 1, wherein a level of ROS is less than 1 when the level in a control is set to 1.

7. A method for producing an oocyte maturation promoter, comprising the steps of:
(1) disrupting adipose tissue-derived stem cells;
(2) filtering a disrupted cell solution obtained in step (1) or a supernatant obtained by centrifuging the disrupted cell solution to prepare a filtrate; and
(3) providing the filtrate obtained in step (2) as a preparation.

8. A method for producing a mature oocyte, comprising culturing an oocyte in presence of the oocyte maturation promoter according to claim 1.

9. An in vitro fertilization method comprising causing an oocyte treated with the oocyte maturation promoter according to claim 1 to coexist with a sperm in vitro.

10. An in vitro fertilization method comprising causing an oocyte and a sperm to coexist in vitro in presence of the oocyte maturation promoter according to claim 1.

11. An embryo transfer method comprising: culturing a fertilized embryo produced by the in vitro fertilization method according to claim 9; and injecting the resulting blastocyst into a uterus of a mammal.

12. Use of the oocyte maturation promoter according to claim 1 as an oocyte quality enhancer.

13. The oocyte maturation promoter according to claim 2, wherein a biological species of the adipose tissue-derived stem cells and a biological species of the oocyte are identical.
